(19) United States
(12) Patent Application Publication
(10) Pub. No.: US 2025/0135046 A1
(43) Pub. Date: May 1, 2025

(54) CHOLECYSTOKININ B RECEPTOR-TARGETED COMPLEX AND A CONTRAST AGENT THEREOF

(71) Applicant: National Atomic Research Institute, Taoyuan (TW)

(72) Inventors: MING-CHENG CHANG, Taoyuan (TW); CHUN-TANG CHEN, Taoyuan (TW); PING-FANG CHIANG, Taoyuan (TW); CHENG-LIANG PENG, Taoyuan (TW)

(21) Appl. No.: 18/495,920

(22) Filed: Oct. 27, 2023

(51) Int. Cl. A61K 51/08; A61P 35/00; C07K 7/06

(52) U.S. Cl. CPC A61K 51/088; A61P 35/00; C07K 7/06; A61K 2121/00; A61K 2123/00

(57) ABSTRACT

Disclosed herein is a drug complex having a synthetic peptide, which has an amino acid sequence of SEQ ID: NO.1; a metal chelator coupled to the synthetic peptide; and a peptide chain disposed between the synthetic peptide and the metal chelator, wherein the peptide chain is composed of plural glutamate molecules.

### SUBMISSION OF SEQUENCE LISTING AS XML FILE

The content of the following submission on XML file is incorporated herein by reference in its entirety: a computer readable form (CRF) of the Sequence Listing (file name: 1100214USIE1_sequence.xml, date recorded: 2023 Sep. 27, size: 4 KB). An identical copy of the Sequence Listing is attached following the specification.

### TECHNICAL FIELD

The present disclosure relates to the field of radiological imaging and diagnosis, in particular to a complex capable of specifically targeting cholecystokinin B receptors.

### BACKGROUND

In a variety of cancers, such as colorectal, pancreatic, ovarian, gastric, thyroid tumors etc., it has been observed that all these tumors are exhibiting an overexpression of cholecystokinin. It is also known that the overexpression of cholecystokinin is related to the development and metastasis of tumor cells. Cholecystokinin has been used as the target in the diagnosis and treatment of tumor imaging and targeted radiotherapy, and it is clinically being used in animal experiments. Drugs targeting cholecystokinin are developed mainly basing on gastrin analogues, which are peptide drugs that can specifically bind to cholecystokinin. However, gastrin peptide drug shows a high degree of binding within kidney, which may hinder the visualization of tumors near kidney, especially when the peptide is used for tumor treatment. One other peptide drug, sCCK8, is also used in the development of drugs for the diagnosis and treatment of cholecystokinin overexpressed tumors. Although the uptake of sCCK8 in kidney is relatively low, sCCK8 contains sulfated tyrosine (Tyr) residues that are easily hydrolyzed. Besides, the sCCK8 sequence contains two methionines (Met) which are susceptible to oxidation during radiolabeling, most likely in vivo. There is still room for improving the development of radioactive antitumor drugs targeting cholecystokinin. In view of this, there is an urgent need in the art for a novel medicine to improve the disadvantages of the prior art.

### SUMMARY

In order to make the readers understand the implication of the disclosure, the summary of the disclosure provides a brief description of the disclosure. The summary of the disclosure is not a complete description of the disclosure, and it is not intended to define the technical characteristics or scope of rights of the present disclosure.

One object of the present disclosure is to provide a cholecystokinin B receptor-targeted complex. The complex includes a synthetic peptide, a metal chelator, a peptide chain and an albumin affinity ligand. The synthetic peptide has the amino acid sequence of SEQ ID: NO. 1 (Ala-Tyr-Gly-Trp-Nle-Asp-Phe). The metal chelator is coupled with the synthetic peptide; and the peptide chain is arranged between the synthetic peptide and the metal chelator, wherein the peptide chain is composed of multiple glutamic acids. The albumin affinity tag is arranged between the peptide chain and the metal chelator. In a specific embodiment of the present disclosure, the peptide chain is composed of at least 6 glutamic acids. In the other embodiment of the present disclosure, the peptide chain has the amino acid sequence of SEQ ID: NO. 2, which is composed of 6 glutamic acids (Glu-Glu-Glu-Glu-Glu-Glu).

In one embodiment, the metal chelator is selected from the group consisting of following materials: 1,4,7,10-tetraazacyclododecane-1,4,7,10-tetraacetic acid (DOTA), 1,4,7-triazacyclononane-1,4,7-triacetic acid (NOTA), 1,4,7-triazacyclononane-1,4-diacetic acid (NODA) and diethylenetriaminepentaacetic acid (DTPA).

In yet another embodiment of the present disclosure, the metal chelator is further labeled with a radioactive nuclide. In addition, in one embodiment, the radioactive nuclide is selected from the group consisting of gallium-66 (Ga-66), gallium-67 (Ga-67), gallium-68 (Ga-68), zirconium-89 (Zr-89), lutetium-177 (Lu-177), indium-111 (In-111), and iodine-123 (I-123). In a specific embodiment, the radioactive nuclide is lutetium-177 (Lu-177).

In one embodiment of the present disclosure, the albumin affinity tag is Lys(-4-TBA)-6-AHA or Lys(-4-TBA)-AMBA. The 4-TBA is the abbreviation of 4-p-Tolylbutyric acid; the 6-AHA is the abbreviation of 6-aminohexanoic acid; the AMBA is the abbreviation of 4-Aminomethylbenzoic acid.

Another aspect of the present disclosure relates to a contrast agent, including the complex described in any one of the above embodiments and a contrast excipient.

Another aspect of the present disclosure relates to the use of the complex described in any of the above-mentioned embodiments in preparation of a pharmaceutical composition for treating and targeting cholecystokinin B receptor-related cancers. In one embodiment, the cancer is a neuroendocrine tumor, pancreatic cancer, esophageal cancer, gastric cancer, liver cancer, colorectal cancer, or medullary thyroid cancer.

### BRIEF DESCRIPTION OF THE DRAWINGS

Those with ordinary knowledge in the technical field of the present invention can fully understand the central concept, adopted technical means and various implementation aspects of the present invention after referring to the following embodiments.

To make the foregoing and other objects, features, advantages, and examples of the present invention more comprehensibly, the drawings are described as follows:

FIG. 1 is the chemical structure of the cholecystokinin B receptor-targeted complexes according to one embodiment of the present disclosure.

FIG. 2 is the results of cell surface adsorption and endocytosis experiments of A431-CCK2R(+) cells to the cholecystokinin B receptor-targeted complex of the present disclosure according to another embodiment of the present disclosure.

FIG. 3 is the results of the Nano SPECT/CT bioimaging experiment of the cholecystokinin B receptor-targeted complex of the present disclosure according to yet another embodiment of the present disclosure.

FIG. 4 is the results of the cholecystokinin B receptor-targeted complex of the present disclosure inhibiting the growth of tumors overexpressing cholecystokinin B receptor according to other embodiments of the present disclosure.

### DETAILED DESCRIPTION

To make the description of the present disclosure more detailed and complete, the following provides an illustrative text description of the implementations and specific examples of the present invention; but the implementations and specific examples of the present disclosure are not limited thereto.

It will be apparent, however, that one or more embodiments may be practiced without these specific details. In other instances, well-known structures and devices are schematically shown in order to simplify the drawing. In the following description and/or scope of patent application, the technical terms used should be interpreted with the usual meanings commonly used by those skilled in the art. For ease of understanding, the same elements in the following embodiments are referred to as the same symbols.

In this specification, the term “about” usually means that the actual value is within plus or minus 10%, 5%, 1% or 0.5% of a specific value or range. The term “about” herein means also that the actual value falls within an acceptable standard error of the mean, as considered by one of ordinary skill in the art to which this invention pertains. Except for the examples, or unless otherwise expressly stated, it should be understood that ranges, amounts, values and percentages used herein are modified by “about”. Therefore, unless otherwise stated, the numerical values or parameters disclosed in this specification and the appended patent claims are approximate numerical values and may be changed as required.

Unless otherwise specified, the “synthetic peptide” disclosed herein includes a specific amino acid sequence including its preserved modification variations, meaning that the substation of the amino acid(s) on the peptide does not affect the activity of the peptide which is the ability to bind to cholecystokinin B receptor of the cancer cells.

The word “individual” or “patient” refers to an animal capable of receiving the complex of the present disclosure. In a preferable implementation, the animal is a mammal, and in particular, is a human.

Several embodiments are disclosed below to illustrate various implementation aspects of the present disclosure, in order to make one skilled in the art of the present disclosure can implement the technical content disclosed in the present disclosure according to the disclosure of this specification. Therefore, the embodiments disclosed below should not be view as to limit the scope of the present disclosure. In addition, all documents cited in this specification are deemed to be fully cited as a part of this specification.

In order to solve the problems existing in the prior art, the present disclosure proposes a novel complex applied in the field of radiotherapy, which possesses higher specificity and stronger therapeutic results. The complex of the present disclosure composes a peptide drug, a glutamic acid peptide chain and albumin affinity tag, which can specifically bind to the receptor to reduce non-specific binding and prevent radiation exposure to normal organs, and therefore, enhance the interaction between therapeutic radioactive nuclide(s) and target cells. In addition to blocking the interaction(s) between cholecystokinin B and its receptor and stopping the growth of the cancer cells, the complex of the present disclosure can also kill cancer cells through the B radiation of the radioactive nuclide(s), which results in a more significant therapeutic effect. Furthermore, by using high sensitivity single photon emission computed tomography (CT) scan in combination with the complex of the present disclosure, monitoring of cancer cell metastasis, disease progress, and curative effect can be achieved in a non-invasive way. The application also obtains good images as shown in the following experimental examples. Besides, the complex of the present disclosure can also be prepared as a kit. The kit can be prepared by the direct labeling method, which increases the convenience of labeling and reduces the chance of personnel exposure to the radiation, and has good stability. It can thus be applied to tumors where the cholecystokinin B receptor is significantly involved, such as neuroendocrine tumors, pancreatic cancer, esophageal cancer, gastric cancer, liver cancer, colorectal cancer, and medullary thyroid cancer, etc., for the diagnosis and treatment as well as imaging and targeted radiotherapy.

### Experiment 1: Cholecystokinin B Receptor-Targeted Complex of the Present Disclosure

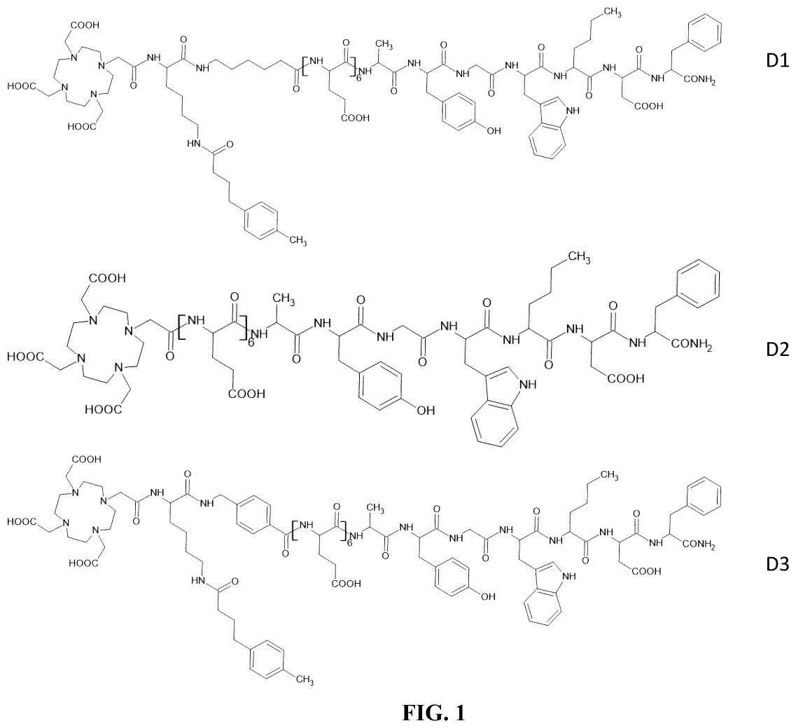

The complex of the present disclosure is synthesized by Zheng-Bai Technology Co., Ltd., and the specific structure and composition of the complex are shown in TABLE 1 and FIG. 1.

TABLE 1

| Name | The Complexes of the present disclosure |
|---|---|
| D1 | DOTA-Lys(-4-TBA)-6-AHA-(Glu)₆-Ala-Tyr-Gly-Trp-Nle-Asp- |
| | Phe-NH₂ |
| D2 | DOTA-(D-Glu)₆-Ala-Tyr-Gly-Trp-Nle-Asp-Phe-NH₂ |
| D3 | DOTA-Lys(-4-TBA)-AMBA-(Glu)₆-Ala-Tyr-Gly-Trp-Nle-Asp- |
| | Phe-NH₂ |

The complex provided by the present disclosure uses albumin affinity tag: Lys(-4-TBA)-6-AHA or Lys(-4-TBA)-AMBA to increase the binding affinity between the drug and the receptor, and by thus, to reduce non-specific binding of the drug to other receptors and radiation exposure to the normal organs, and to prolong the drug circulation time in the blood to increase the cumulation of the drug in the tumors. As a result, it can enhance the interaction(s) between the therapeutic radioactive nuclide and the target tumor cells.

### Experiment 2: Radioactive Nuclide Indium-111 Labeling the Complexes of the Present Disclosure and the Drug Stability Tests

Firstly, the radioactive nuclide indium-111 labeling conditions for complexes D1 to D3 of the present disclosure are optimized. Six different conditions have been tested for the radioactive labeling efficiencies, including the use of the molar ratio of radioactive nuclide indium-111 and the complex of the present disclosure of 1:20 and 1:30 under 5 different activity concentrations of radioactive nuclide indium-111. The detailed conditions are listed in TABLE 2. According to the experiment data, with 1.5 ug of the complex, the optimized molar ratio of radioactive nuclide indium-111 and the complex is 1:30, which can obtain the highest radioactive labeling efficiency. After labeling, the radioactive nuclide labeled complexes are analyzed by using Radio-TLC. The results indicate that the labeling efficiency for the radioactive nuclide indium-111 labeling the complexes D1, D2, and D3 are 97.4+1.5%, 96.4+1.2%, and 96.4+1.2%, respectively. The indium-111 labeling efficiency can reach higher than 90% for each complex.

TABLE 2

| Indium-111 | | In-111:D1 | | In-111:D2 | | In-111:D3 |
|---|---|---|---|---|---|---|
| Activity | D1 | (molar | D2 | (molar | D3 | (molar |
| Concentration | (ug) | ratio) | (ug) | ratio) | (ug) | ratio) |
| 1 mCi | 1 | 1:19.38 | 1 | 1:23.43 | 1 | 1:19.57 |
| 1 mCi | 1.5 | 1:29.07 | 1.25 | 1:29.29 | 1.5 | 1:29.36 |
| 2 mCi | 3 | 1:29.07 | 2.5 | 1:29.29 | 3 | 1:29.36 |
| 3 mCi | 4.5 | 1:29.07 | 3.75 | 1:29.29 | 4.5 | 1:29.36 |
| 4 mCi | 6 | 1:29.07 | 5 | 1:29.29 | 6 | 1:29.36 |
| 5 mCi | 7.5 | 1:29.07 | 6.25 | 1:29.29 | 7.5 | 1:29.36 |

Then, the complexes D1, D2, and D3 of the present disclosure (the cholecystokinin B receptor-targeted complexes) labeled with the radioactive nuclide In-111 are placed in the normal saline and kept in a 4° C. refrigerator to determine the radioactive stability with respect to different periods of time. The results show that, under 4° C. normal saline in vitro condition for 48 hours, the radioactive nuclide In-111 labeled D1 and D3 can still possess the stability higher than 90%, while In-111 labeled D2 has relatively lower radioactive stability of 84.1+1.1%.

### Experiment 3: Radioactive Nuclide Lutetium-177 Labeling the Complexes of the Present Disclosure and Drug Stability Tests

The radioactive nuclide lutetium-177 labeling conditions for complexes DI to D3 of the present disclosure are optimized. Seven different conditions have been tested for the radioactive labeling efficiencies separately, including the use of the molar ratio of radioactive nuclide lutetium-177 and the cholecystokinin B receptor-targeted complexes of the present disclosure of 1:10, 1:30 and 1:60 under 5 different activity concentrations of radioactive nuclide lutetium-177. The detailed conditions are listed in TABLE 3. According to the experiment data, with 4 μg of the complex, the optimized molar ratio of radioactive nuclide lutetium-177 and the complex is 1:60, which can obtain the highest radioactive labeling efficiency. After labeling, the radioactive nuclide labeled complexes are analyzed by using Radio-TLC. The results indicate that the labeling efficiency for the radioactive nuclide lutetium-177 labeling the complexes D1, D2, and D3 are 96.3±1.3%, 98.4±1.2%, and 99.2±0.7%, respectively. The lutetium-177 labeling efficiency can reach higher than 90% for each complex.

Then, the complexes D1, D2, and D3 of the present disclosure (the cholecystokinin B receptor-targeted complexes) labeled with the radioactive nuclide Lu-177 are placed in the normal saline and kept in a 4° C. refrigerator to determine the radioactive stability with respect to different periods of time. The results show that, under 4° C. normal saline in vitro condition for 48 hours, the radioactive nuclide Lu-177 labeled D1 and D3 can still possess the stability higher than 90%, while Lu-177 labeled D2 has relatively lower radioactive stability of 78.8±1.0%.

TABLE 3

| Lutetium-177 | | | | | | |
|---|---|---|---|---|---|---|
| Activity | D1 | Lu-177:D1 | D2 | Lu-177:D2 | D3 | Lu-177:D3 |
| Concentration | (ug) | (molar ratio) | (ug) | (molar ratio) | (ug) | (molar ratio) |
| 1 mCi | 1 | 1:8.14 | 1 | 1:9.8 | 1 | 1:8.2 |
| 1 mCi | 4 | 1:32.5 | 3 | 1:29.52 | 4 | 1:32.88 |
| 2 mCi | 8 | 1:32.5 | 6 | 1:29.52 | 8 | 1:32.88 |
| 3 mCi | 12 | 1:32.5 | 9 | 1:29.52 | 12 | 1:32.88 |
| 4 mCi | 16 | 1:32.5 | 12 | 1:29.52 | 16 | 1:32.88 |
| 5 mCi | 20 | 1:32.5 | 15 | 1:29.52 | 20 | 1:32.88 |
| 1 mCi | 4 | 1:60 | 3 | 1:60 | 4 | 1:60 |

### Experiment 4: Cell Surface Adsorption and Endocytosis Experiments of Radioactive Nuclide Lutetium-177 Labeled Complexes

In order to verify that the radioactive nuclide labeled complexes of the present disclosure can be used as tumor targeted therapy drugs through the interaction(s) with cholecystokinin B receptor, the cellular uptake experiments for the drugs are performed. Firstly, the complexes D1 to D3 are radioactive labeled with lutetium-177 to obtain the radioactive nuclide lutetium-177 labeled complexes D1, D2 and D3 (Lu-177-D1, Lu-177-D2, Lu-177-D3 hereafter). Then, add Lu-177-D1, Lu-177-D2 and Lu-177-D3 into A431-CCK2R⁽⁺⁾ and A431-CCK2R⁽⁻⁾ cells. More specifically, firstly, the A431-CCK2R⁽⁺⁾ and A431-CCK2R⁽⁻⁾ cells are respectively cultured in a 6-well plate with cell density of 1×106 cells/well. After 24 hours cultivation, add 2 μCi of Lu-177-D1, Lu-177-D2, and Lu-177-D3 into the well (each well contains 1 ml medium) respectively, and resume to cultivate under 37° C. for another 1 hour and 4 hours, three repeats (n=3) for each drug and time. Then, use Gamma-counter to determine the radioactivity, and calculate the fraction of radioactive drugs (Lu-177-D1, Lu-177-D2, and Lu-177-D3) accumulated in cells. The results indicate that, after adding Lu-177-D1, Lu-177-D2, and Lu-177-D3 respectively into wells having A431-CCK2R⁽⁻⁾ cell culture, and resuming to cultivate for another 1 hour, the results for the cell surface adsorption drug amount of each complex is 1.08±0.49, 3.42±0.21, and 0.59±0.01 (% IA/10⁶ cells), respectively. Even when resuming cultivation time is prolonged to 4 hours, the cell surface adsorption drug amount for each complex does not show significantly increasing. The results are 1.04±0.35, 3.24±0.15, and 0.55±0.09 (% IA/10⁶ cells) for Lu-177-D1, Lu-177-D2, and Lu-177-D3 respectively. Further, the results of the cell endocytosis experiments for Lu-177-D1, Lu-177-D2, and Lu-177-D3 are analyzed. After 1 hour treatment, the results for the cell endocytosis drug amount of each complex (Lu-177-D1, Lu-177-D2, and Lu-177-D3, respectively) are 0.32±0.10, 1.26±0.06, and 0.25±0.04 (% IA/10⁶ cells) respectively. Similarly, the results for the 4 hours treatment for each complex does not show significantly increasing, they are 0.15±0.18, 1.29±0.01, and 0.17±0.03 (% IA/10⁶ cells).

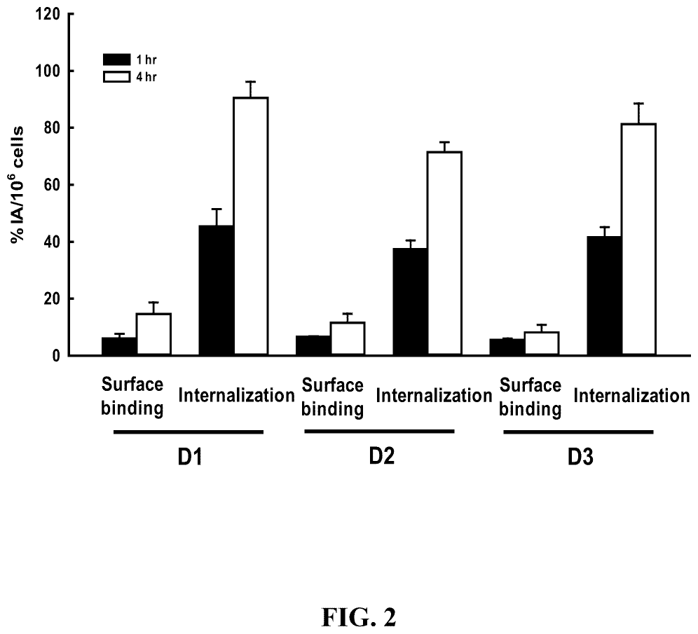

The above results indicate that the radioactive nuclide labeled complexes of the present disclosure cannot enter into A431-CCK2R⁽⁻⁾ cell since the cell does not express cholecystokinin B receptor. By contrast, when adding the radioactive nuclide labeled complexes of the present disclosure into A431-CCK2R⁽⁺⁾ cell, which can overexpress cholecystokinin B receptor, the cell surface adsorption and endocytosis of the drugs can be observed. More specifically, after adding Lu-177-D1, Lu-177-D2, and Lu-177-D3 respectively into the cell culture containing A431-CCK2R⁽⁺⁾ cells, and resuming culturing for 1 hour, the results for the cell surface adsorption drug amount of each complex are 5.93±1.68, 6.60±0.17, and 5.51±0.45 (% IA/10⁶ cells). Besides, when the drug treatment time is prolonged from 1 hour to 4 hours, the results for the cell surface adsorption drug amount of each complex are significantly increased, which are 14.57±4.06, 11.51±3.16, and 8.11±2.65 (% IA/10⁶ cells) with respect to Lu-177-D1, Lu-177-D2, and Lu-177-D3. Further, the results for the cell endocytosis experiments of Lu-177-D1, Lu-177-D2, and Lu-177-D3 are analyzed. After 1 hour drug treatment, the results for the cell endocytosis drug amount of each complex (Lu-177-D1, Lu-177-D2, and Lu-177-D3, respectively) are 45.32±6.12, 37.37±3.04, and 41.58±3.46 (% IA/10⁶ cells) respectively. While when the drug treatment time is prolonged from 1 hour to 4 hours, the results for the cell endocytosis drug amount are significantly increasing to 90.49±5.65, 71.44±3.52, and 81.26±7.27 (% IA/10⁶ cells). These results indicate that all of the radioactive nuclide Lu-177 labeled complexes, D1, D2, and D3, exhibit highly binding affinity with the cell which overexpresses cholecystokinin B receptor, and can be uptaken into the cholecystokinin B receptor-overexpressing cells through specifically binding to the cholecystokinin B receptor (Refer to FIG. 2).

### Experiment 5: The Radioactive Nuclide Lutetium-177 Labeled Complex of the Present Disclosure Applied in the Treatment of Tumor Mice

Establishment of tumor animal model: Five-to six-week-old male experimental nude mice are purchased from the National Laboratory Animal Center (NLAC, TAIWAN) and bred in the animal facility. All animal experiment procedures are performed in accordance with the protocols approved by Institutional Animal Care and Use Committee (IACUC, TAIWAN). The experimental mice are kept at a temperature of 21-23° C., and a 12-hour light-dark cycle are used as the basis for the animal's physiological cycle.

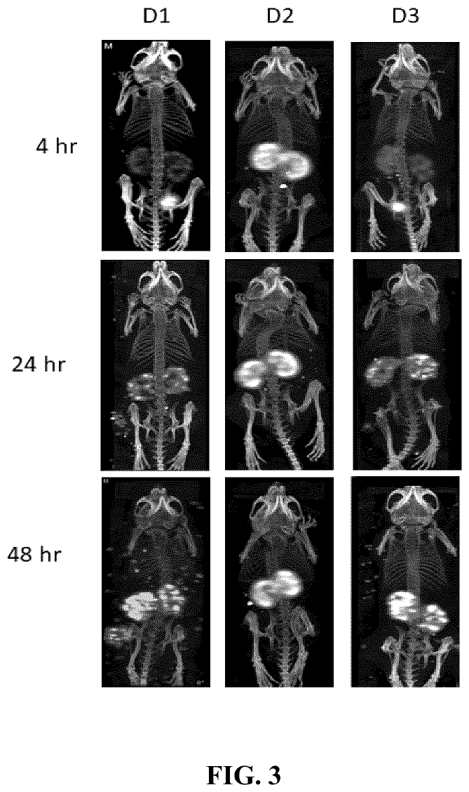

A431-CCK2R⁽⁻⁾ and A431-CCK2R⁽⁺⁾ cells (2×10⁶ cells) are inoculated in the left and right hind limbs of the mice. After the tumors growing to 200-300 mm³, the radioactive labeled complexes Lu-177-D1, Lu-177-D2, and Lu-177-D3 are used as radiotherapy drugs and start the treatments. The cholecystokinin B receptor-targeted radiotherapy drugs L-177-D1, Lu-177-D2, and Lu-177-D3 are administrated to the mice respectively by tail vein injection. After different time periods of administration, such as 4, 24, and 48 hours, the coronal sections of three tumor mice are obtained (Refer to FIG. 3). By showing the comparison of NanoSPECT and CT fusion images at different time points, the accumulation of these radiotherapy drugs in the tumor tissue can be observed. Then measure and record the tumor size twice a week. The experimental results show that the drug Lu-177-D2 complex, which is currently undergoing clinical trials, does not show a significant drug accumulation in the imaging results of the existing three time points, and is mainly accumulated in kidney. In comparison, the Lu-177-D1 and Lu-177-D3 complexes of the present disclosure are observed to be able to accumulate in the tumor site where the cholecystokinin B receptor is overexpressed after starting the animal experiment for 24 hours. In addition, the drug accumulation in the tumor site is observed to be more obvious as the treatment time increases in both Lu-177-D1 and Lu-177-D3 complexes treatment, and can last for at least 48 hours. These data show that the radiotherapeutic drugs Lu-177-D1 and Lu-177-D3 complexes can significantly accumulate in cholecystokinin B receptor overexpressing tumor cells.

### Experiment 6: Radioactive Nuclide Lutetium-177 Labeled Complexes of the Present Disclosure Inhibit the Growth of the Tumor Overexpressing Cholecystokinin B Receptor

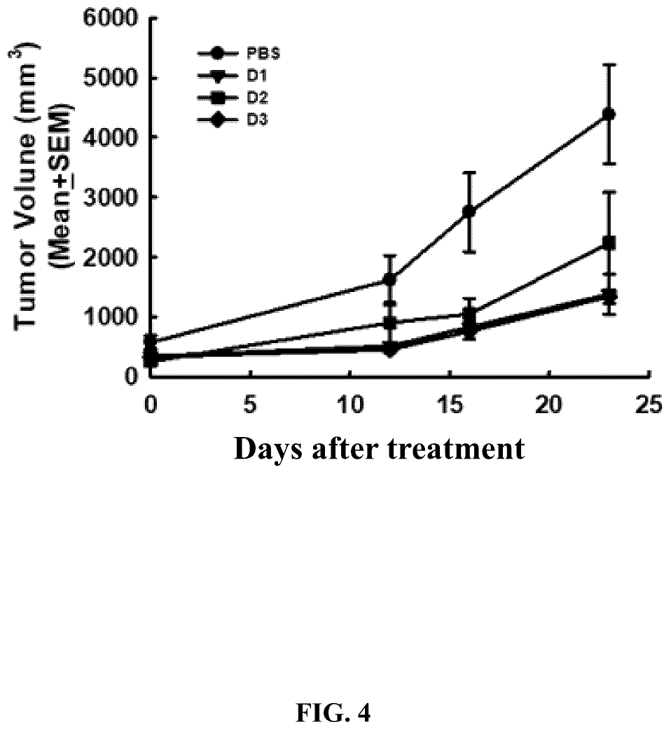

After seven days of tumor injection, experimental animals start to receive radiotherapy drugs Lu-177-D1 and Lu-177-D3 treatment. After twenty-three days of treatment, the tumor volume has grown to 3.8-4.1 times larger compared with the initial tumor volume (Experiment of Lu-177-D1 complex=>tumor volume: D0=352.1±44.3 mm³, D23=1384.7±333.1 mm³. Experiment of Lu-177-D3 complex=>tumor volume: D0=308.8±55.6 mm³, D23=1162.6±75.9 mm³). The differences between the results of the two drugs are not statistically significant. In addition, the tumor volume in tumor-bearing mice that are only treated with normal saline have grown to about 7.6 times larger compared with the initial tumor volume (Experiment of normal saline=>tumor volume: D0=575.2±112.0 mm³, D23=4386.0±828.7 mm³). Comparing with the Lu-177-D2 complex treatment (Experiment of Lu-177-D2 complex=>tumor volume: D0=258.6.2±32.3 mm³, D23=2237.1±846.3 mm³), the Lu-177-D1 and Lu-177-D3 complexes exhibit significantly better treatment results. The tumor volume in the Lu-177-D1 or Lu-177-D3 complexes treatment experiment is only 51.9%˜61.81% of the tumor volume in the Lu-177-D2 complex treatment experiment, please refer to the results shown in FIG. 4. According to the above-mentioned experiment results, it is obvious that the radioactive nuclide labeled complexes of the present disclosure exhibit significant treatment results in treating the cholecystokinin B receptor overexpression tumor, and have the potential to become a novel peptide drug in diagnosis and treatment of cancers which overexpressing cholecystokinin B receptor.

What is claimed is:

1. A cholecystokinin B receptor-targeted complex, comprising:
a synthetic peptide, comprising the amino acid sequence of SEQ ID NO: 1 (Ala-Tyr-Gly-Trp-Nle-Asp-Phe);
a metal chelator, coupling to the synthetic peptide;
a peptide chain, disposing between the synthetic peptide and the metal chelator, wherein the peptide chain includes the amino acid sequence of SEQ ID NO: 2 (Glu-Glu-Glu-Glu-Glu-Glu), consisting of 6 glutamic acids; and
an albumin affinity tag, disposing between the peptide chain and the metal chelator; wherein the albumin affinity substance is Lys(-4-TBA)-6-AHA or Lys(-4-TBA)-AMBA.

2. The cholecystokinin B receptor-targeted complex of claim 1, wherein the metal chelator is selected from the group consisting of: 1,4,7,10-tetraazacyclododecane-1,4,7,10-tetraacetic acid (DOTA), 1,4,7-triazacyclononane-1,4,7-triacetic acid (NOTA), 1,4,7-triazacyclononane-1,4-diacetic acid (NODA) and diethylenetriaminepentaacetic acid (DTPA).

3. The cholecystokinin B receptor-targeted complex of claim 1, further including a radioactive nuclide labeled on the metal chelator.

4. The cholecystokinin B receptor-targeted complex of claim 3, wherein the radioactive nuclide is selected from the group consisting of: gallium-66 (Ga-66), gallium-67 (Ga-67), gallium-68 (Ga-68), zirconium-89 (Zr-89), lutetium-177 (Lu-177), indium-111 (In-111) and iodine-123 (I-123).

5. The cholecystokinin B receptor-targeted complex of claim 3, wherein the radioactive nuclide is lutetium-177 (Lu-177).

6. A method of preparing a pharmaceutical composition for treating or diagnosing a disease using the cholecystokinin B receptor-targeted complex of claim 1, wherein the disease is a cancer overexpressing or highly expressing cholecystokinin B receptor.

7. A method of claim 6, wherein the cancer is neuroendocrine tumor, pancreatic cancer, esophageal cancer, gastric cancer, liver cancer, colorectal cancer or medullary thyroid cancer.

8. A contrast agent, comprising the cholecystokinin B receptor-targeted complex of claim 1; and a contrast excipient.


SEQUENCE LISTING (2 sequences)

SEQ ID NO: 1 - protein, 7 aa, synthetic construct
  REGION 1..7: note = Synthetic peptide
  MOD_RES 5: note = The 5th residue Xaa is Nle
  MOD_RES 7: note = AMIDATION at C-terminal
AYGWXDF

SEQ ID NO: 2 - protein, 6 aa, synthetic construct
  REGION 1..6: note = peptide chain
EEEEEE